(19) United States
(12) Patent Application Publication
(10) Pub. No.: US 2006/0210490 A1
(43) Pub. Date: Sep. 21, 2006

(54) Confection for teeth whitening and oral hygiene

(76) Inventors: Heryk P. Jakubowski, Forest Hills, NY (US); Hanna Jakubowski, Forest Hills, NY (US)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/384,774

(22) Filed: Mar. 20, 2006

Related U.S. Application Data
(63) Continuation-in-part of application No. 11/084,468, filed on Mar. 19, 2005.

(51) Int. Cl. A61K 8/20; A61K 9/68

(52) U.S. Cl. 424/48; 424/53

(57) ABSTRACT

Confections for teeth whitening and oral hygiene that comprise a first component including a first bulking agent, an organic acid, and a peroxide, and a second component including a white pigment dissolved in an alkaline solution and combined with a second bulking agent. The first component forms hydrogen peroxide, a bleaching agent, as the confection dissolves in saliva. The hydrogen peroxide cleans teeth and removes stains. The second component acts as a whitening agent to cover any stains that were not removed by the hydrogen peroxide. In one embodiment of the invention, supplements are added to the confection that function as substitutes for stain inducing activities, such as drinking coffee and smoking cigarettes, thereby preventing further staining of the teeth.

### PRIORITY

This application is a continuation-in-part of application Ser. No. 11/084,468 filed Mar. 19, 2005.

### FIELD OF THE INVENTION

The present invention generally relates to dentifrices, and in particular to confections for teeth whitening and oral hygiene.

### BACKGROUND OF THE INVENTION

Teeth whitening has become very popular in recent years. Dental procedures, home kits, toothpastes, and even chewing gums have been developed to whiten teeth. These methods introduce a bleaching agent to the surface of the teeth in order to whiten them. One of the more common bleaching agents employed with these methods is hydrogen peroxide.

Dental procedures for bleaching teeth are perhaps the most effective method of whitening teeth. The use of high concentrations of relatively harsh chemicals precisely applied by a trained professional are responsible for the effectiveness of this whitening procedure. However, there are several drawbacks associated with dental whitening procedures. Dental procedures are time consuming and require making an appointment with the trained professional. Often times dental procedures require follow-up visits and can be very expensive. The high concentrations of relatively harsh chemicals are also undesirable. Furthermore, dental procedures for teeth whitening are negatively associated with teeth sensitivity, sore/injured gums, and an unpleasant taste.

While not as effective as dental procedures, home kits have become a popular form of bleaching teeth. The home kits use a lower concentration of bleaching agent than the dental procedures. However, the home kits require applying the bleaching agent directly to the teeth either by painting the bleaching agent onto the surface of the teeth or through the use of a bleaching tray or whitening strips. The home kits require several days worth of application, and often have side effects similar to those associated with dental procedures. Furthermore, the bleaching trays and whitening strips are often uncomfortable and irritating. Many home kits require application of the bleaching agent multiple times a day. Whitening strips require the strips to be left in the mouth for several hours. These inconveniences are a serious deficiency in home kits.

Toothpastes have also been used to bleach teeth, however, toothpastes rely heavily on the mechanical action of brushing to effectively whiten the teeth. The brushing is generally relied upon to remove the stains, while other additives in the toothpaste work to whiten the teeth. Toothpastes generally are in contact with the teeth for only a short duration of time making them poor whitening agents. Furthermore, toothpastes require a toothbrush and a water source which are generally not readily available outside a person's home. Although whitening agents such as titanium white are often present in toothpastes, they usually function to give the toothpaste its white color rather than to act as a whitener for the teeth.

Finally, chewing gums have also been used as whitening agents for teeth. These gums employ surfactants as an active cleaning agent. Often times chewing gums rely on abrasives within the gum to mechanically remove stains while being chewed. Due to the low concentration of bleaching agents, there is a necessity for repeated usage. Unfortunately, repeated usage of chewing gum is negatively associated with teeth loosening, bruxism, and Temporo-Mandibular Joint Syndrome (TMJ). Many people are unable to chew gum as a result of dental work or prosthetics. Furthermore, chewing gums require a means of disposing the gum which is not always readily available.

To this end, applicant has invented a confection for teeth whitening and oral hygiene described in U.S. patent application Ser. No. 11/084,468. The confection comprises a first component including a first bulking agent, an organic acid, and a peroxide, and a second component including a white pigment dissolved in an alkaline solution and combined with a second bulking agent. The first component forms hydrogen peroxide, a bleaching agent, as the confection dissolves in saliva. The hydrogen peroxide cleans the teeth and removes stains therefrom. The second component acts as a whitening agent to cover any stains that were not removed by the hydrogen peroxide.

However, although the confection described above whitened teeth and provided some degree of oral hygiene, a need still existed for a confection for teeth whitening and oral hygiene that also freshened breath. Additionally, the applicant has discovered that chocolate may be used as the bulking agent which has the added benefit of being chewed and not sucked. Furthermore, ingredients such as caffeine, and/or nicotine may be added to the confection so that the confection can prevent or reduce staining by functioning as a substitute for teeth staining activities such as smoking and coffee drinking.

### OBJECTS AND SUMMARY OF THE INVENTION

It is therefore an object of the present invention to provide new and improved confections for teeth whitening and oral hygiene.

Another object of the present invention is to provide new and improved confections for teeth whitening and oral hygiene that are also breath fresheners.

Yet another object of the present invention is to provide new and improved confections employing low concentrations of bleaching agents.

Still another object of the present invention is to provide new and improved confections that do not require direct application of bleaching agent or a mechanical action to whiten teeth.

A further object of the present invention is to provide new and improved confections that prevent or reduce further staining of teeth.

Briefly, in accordance with the present invention, these and other objects are attained by providing confections for teeth whitening and oral hygiene that comprise a first component including a first bulking agent, an organic acid, and a peroxide, and a second component including a white pigment dissolved in an alkaline solution and combined with a second bulking agent. The first component forms hydrogen peroxide, a bleaching agent, as the confection dissolves in saliva. The hydrogen peroxide cleans teeth and removes stains. The second component acts as a whitening agent to cover any stains that were not removed by the hydrogen peroxide.

In a first embodiment, the confection is a hard sucking candy. The hard shell of the candy is formed by the first component whose bulking agent is a sugar-free sweetener, such as Isomalt. The inside of the candy is formed by the second component and has a softer consistency.

In a second embodiment, the confection is a chewable candy. The chewable consistency of the candy is attributable to the first and second bulking agents which are formed of a substance such as chocolate. In this embodiment, the first and second components are spread homogenously throughout the confection.

In a third embodiment, in addition to the first and second components, the confection contains ingredients that can be consumed as substitutes for foods and chemicals that stain teeth. In this manner, the confection both whitens teeth and minimizes further staining.

### BRIEF DESCRIPTION OF THE DRAWINGS

A more complete appreciation of the present invention and many of the attendant advantages thereof will be readily understood by reference to the following detailed description when taken in conjunction with the accompanying drawing, in which:

FIG. 1 is a front view partially broken away of a first embodiment of a confection for teeth whitening and oral hygiene according to the present invention.

### DETAILED DESCRIPTION OF THE INVENTION

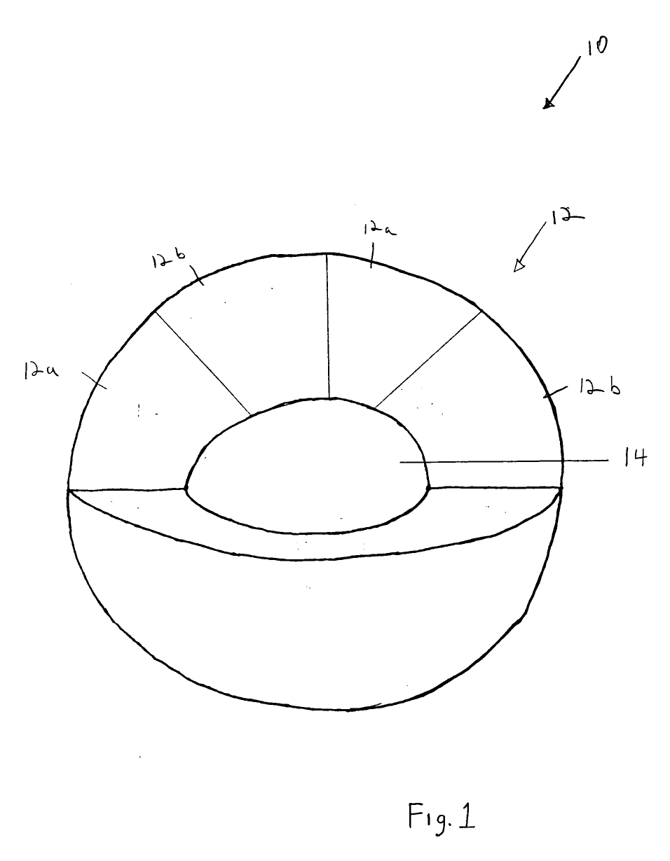

Referring now to the drawings in which like reference characters designate identical or corresponding parts throughout the several views, a preferred embodiment of the present invention will now be described with reference to FIG. 1.

A confection for teeth whitening and oral hygiene, generally designated 10, is formed of two components. The first component, forming an outer layer 12 of the confection, includes a bulking agent, an organic acid, and a peroxide. The bulking agent is a sweetener that is preferably sugar-free, such as a sugar derivative. Isomalt (an equimolar composition of 6-0-alpha-D-glucopyranosido-D-sorbitol (1,6-GPS) and 1-0-alpha-D-glucopyranosido-D-mannitol-dihydrate (1,1-GPM-dihydrate)) is the preferred bulking agent. The bulking agent serves to give the candy its shape and also to act as a buffer between the organic acid and the peroxide. The bulking agent prevents the organic acid and the peroxide from reacting with each other prematurely.

The organic acid is preferably citric acid, although any organic acid or mixtures of organic acid may be used. The peroxide is preferably calcium peroxide. In a preferred embodiment, calcium peroxide concentrations comprise of 0.5% to 5% by the weight of the confection. Citric acid concentrations comprise 1% to 5% by weight of the confection. It should be noted that calcium peroxide is water insoluble which permits the confection to have a longer shelf-life due to its stability. Additionally, surfactants such as sodium laurel phosphate may be added to further aid in mixing the organic acid and the peroxide. Other flavorings may also be added to the confection to improve its flavor and to aid in masking bad breath. It is understood, that the proportions of the ingredients may be adjusted to suit the specific tastes and potency of the confection.

As the confection is sucked, the outer layer 12 comes into contact with saliva and the bulking agent begins to dissolve. The citric acid and the calcium peroxide are released in small consistent bursts and react to produce hydrogen peroxide. The reaction is as follows:
C₆H₈O₇+CaO₂→C₆H₆O₇Ca+H₂O₂
It is understood that if a different organic acid is used the reaction would be different, but it would still result in the production of hydrogen peroxide as a byproduct.

Hydrogen peroxide is a bleaching agent and an oxidant. The hydrogen peroxide bleaches the stains on the teeth and oxidizes protein pellicles present on the surface of the teeth. The hydrogen peroxide also functions to promote oral hygiene by oxidizing odor causing bacteria and disrupting fermentation of food particles by bacteria responsible for tooth decay. The sweet flavor of the bulking agent stimulates saliva secretion. The increase in saliva works to soften calcium deposits (tartar and calculus) further cleaning the teeth and ridding them of stain.

The second component of the confection, forming an inner layer 14, includes a white pigment dissolved in an alkaline solution and combined with a second bulking agent. Titanium white (TiO₂) is a white pigment that is used to whiten the teeth in the present invention. The titanium white has a concentration of 0.5% to 5% by weight of the confection. Titanium white is generally insoluble in water allowing for a longer shelf-life due to its stability. However, titanium white is soluble in an alkaline solution. The alkaline solution is preferably sodium bicarbonate. The sodium bicarbonate has a concentration of between 0.5% to 5% by weight of the confection. In the preferred embodiment, flavorings and sweeteners such as peppermint and cinnamon oil are also added to the second component, as well as a bulking agent. The bulking agent for the second component is organic gum such as arabic or acacia gum.

Once the outer layer 12 has dissolved, the inner layer 14 comes into contact with saliva and it too begins to dissolve into the mouth. The titanium white coats and masks the teeth covering any stains that were not removed by the hydrogen peroxide. The titanium white also fills small fissures and depressions of the teeth, increasing their apparent whiteness. The flavorings and sweeteners freshen breath and contribute to the overall feeling of cleanliness. The bulking agent thickens the solution of sodium bicarbonate and titanium white increasing the contact and reaction time with the teeth for an improved result. Additionally, stimulation of the taste buds will occur by the addition of the citric acid to the solution resulting in formation of effervescence (C₂O₂ dissolved in water). Increased hydration of the mouth caused by sucking on candy will provide relief from dry mouth symptoms. Increased salivation will help with elimination of the bacteria responsible for tooth decay and bad odor. Flavorings such as Tannis (tanic acid—bitter tasting), peppermint, and/or cloves may be added to increase saliva excretions and to enhance mixing of dissolved components.

It is understood that the first and second components may be mixed homogenously throughout the confection, rather than arranged so that they form an outer layer 12 and an inner layer 14. Homogenously mixing the components throughout the confection lends itself to a very simplified production process. However, the layering arrangement within the sucking candy is most effective because it allows the hydrogen peroxide to clean and bleach the teeth before they are coated with titanium white. Furthermore, the layering arrangement minimizes any interference with the chemical reactions.

In the preferred embodiment, the outer layer 12 is made up of two subcomponents. The first subcomponent 12a includes the bulking agent and the organic acid. The second subcomponent 12b includes the bulking agent and the peroxide. Although the first and second subcomponents make up the outer layer, they remain distinct from each other and are not homogenous. This ensures that the organic acid and the peroxide will not prematurely react as a result of environmental conditions. It is understood that the subcomponents may be arranged in a variety of ways and are not limited to the arrangement shown in FIG. 1.

The confection may be produced as a single unit and used in conjunction with an ultrasonic appliance to clean removable dental appliances. Rather than being consumed by an individual, the confection is placed into a container containing a solution. The dental appliance is also submerged in the solution. When ultrasonic waves are introduced into the solution, the confection dissolves and cleans and whitens the dental appliance in the same manner as described above.

In a second embodiment, both the first and second bulking agents are chocolate. This results in a chewable confection rather than a hard sucking candy of the first embodiment. There are many advantages to a chewable confection. Chewing the confection causes the first and second components to be spread to the entire mouth. Sucking the hard candy may result in the first and second components not reaching the front of the teeth if the consumer swallows the released components immediately and is not careful to direct the components to the front of the teeth. Additionally, the chocolate releases the components all at once rather than in small increments. The chocolate is preferably high quality chocolate with a high cocoa content of 50% and above. Another advantage is that chocolate has anti-cavity properties, especially those with low sugar content. The first and second components are homogenously mixed throughout the chocolate. In a variation of this embodiment, the first and second components, namely calcium peroxide and citric acid and alkaline containing titanium white and sodium bicarbonate, will be formed into regular or sugar free sour candy chips using Isomalt as a bulking agent and mixed into chocolate. As the chocolate is chewed and dissolves the chips are crushed releasing bursts of the chemicals. This results in speeds the reaction that results in production of hydrogen peroxide as a bleaching agent and dissolves titanium white to cover the surfaces of the teeth thereby increasing the apparent whiteness of the teeth.

The chocolate may be infused with 7 mg of zinc or zinc compounds, such as zinc chelate, zinc liposome, zinc citrate and liquid zinc, per serving of 25 to 30 mg (typical serving size is two squares or block of chocolate weighting 100 G). The addition of a zinc compound neutralizes bad breath rather than masking it. Other substances with similar properties may also be used, such as chromium compounds. As the chocolate melts, the zinc compound passes through the esophagus neutralizing violate sulfur compounds present in the esophagus and in the stomach. This neutralizes bad breath at its source.

In a third embodiment, the confection not only whitens teeth and promotes oral hygiene, but it also acts as a substitute for teeth staining consumer products such as coffee and cigarettes. This is achieved by adding supplements to the chocolate to give it additional functionality.

Caffeine, in the amount of 115 mg per serving which is the typical amount present in the 5 oz cup of coffee, may be added to the chocolate. The highly caffeinated chocolate can be used as a replacement for coffee that also whitens teeth and freshens breath. Substituting the confection for coffee helps reduce stain on teeth as coffee is often times responsible for the stain. The confection not only whitens teeth and freshens breath, but also functions to prevent additional staining.

Nicotine may also be added to the chocolate to help smokers fight nicotine withdrawal. Smoking is a cause of stain on teeth. The chocolate reinforced with nicotine may be used to reduce the number of cigarettes a smoker smokes while at the same time whitening the smokers teeth. Approximately 0.005 mg. to 2 mg of nicotine should be added per serving of chocolate.

It is understood that the zinc, caffeine, and nicotine mentioned above may be added to chocolate without the first and second components discussed above for a confection that may be used as a substitute for stain forming activities, but does not whiten teeth.

The description above indicates that a great degree of flexibility is offered in terms of the present invention. Although the present invention has been described in considerable detail with reference to certain preferred versions thereof, other versions are possible. Therefore, the spirit and scope of the appended claims should not be limited to the description of the preferred versions contained herein.

We claim:

1. A confection for teeth whitening and oral hygiene comprising:
a first component including a first bulking agent, an organic acid, and a peroxide; and
a second component including a white pigment dissolved in an alkaline solution and combined with a second bulking agent.

2. The confection of claim 1, wherein said first and second components are homogenous.

3. The confection of claim 1, wherein said confection further comprises:
an outer layer including said first component and an inner layer including said second component.

4. The confection of claim 3, wherein said outer layer comprises:
a first subcomponent including said first bulking agent and said organic acid;
a second subcomponent including said first bulking agent and said peroxide; and
wherein said first and second subcomponents remain distinct within said outer layer.

5. The confection of claim 1, wherein said first bulking agent is a sweetener.

6. The confection of claim 5, wherein said first bulking agent is a sugar-free sweetener.

7. The confection of claim 6, wherein said first bulking agent is an equimolar composition of 6-0-alpha-D-glucopyranosido-D-sorbitol (1,6-GPS) and 1-0-alpha-D-glucopyranosido-D-mannitol-dihydrate (1,1-GPM-dihydrate).

8. The confection of claim 1, wherein said organic acid is citric acid.

9. The confection of claim 1, wherein said organic acid comprises a combination of organic acids.

10. The confection of claim 1, wherein said peroxide is calcium peroxide.

11. The confection of claim 1, wherein said first component further comprises flavorings.

12. The confection of claim 1, wherein said first component further comprises natural or artificial sweeteners or both.

13. The confection of claim 1, wherein said first component further comprises a surfactant.

14. The confection of claim 13, wherein said surfactant is sodium laurel phosphate.

15. The confection of claim 1, wherein said peroxide is used in a concentration of 0.5% to 5.0% by weight of the confection.

16. The confection of claim 1, wherein said organic acid is used in a concentration of 1% to 5% of the confection.

17. The confection of claim 1, wherein said white pigment is titanium white.

18. The confection of claim 1, wherein said alkaline solution is sodium bicarbonate.

19. The confection of claim 1, wherein said second component further comprises flavorings or sweeteners or both.

20. The confection of claim 1, wherein said second bulking agent is organic gum.

21. The confection of claim 20, wherein said organic gum is arabic or acacia gum.

22. The confection of claim 1, wherein said first and second components are homogenously mixed in chocolate.

23. The confection of claim 1, wherein said first and second bulking agents are chocolate.

24. The confection of claim 23, wherein said first and second components are homogenously mixed throughout said confection.

25. The confection of claim 1, further comprising a zinc compound.

26. The confection of claim 1, further comprising caffeine.

27. The confection of claim 1, further comprising nicotine.

28. Chocolate for freshening breath comprising cocoa and zinc.

29. The chocolate of claim 28, wherein the amount of zinc added to said chocolate is 7 mg of zinc compounds per serving of 25 to 30 mg of chocolate.

30. The chocolate of claim 28, further comprising caffeine.

31. The chocolate of claim 28, further comprising nicotine.
